(19) United States
(12) Patent Application Publication
(10) Pub. No.: US 2011/0271559 A1
(43) Pub. Date: Nov. 10, 2011

(54) FOOTWEAR ORTHOSIS

(75) Inventors: Franco SALVATELLI, Civitanova Marche (IT); Massimo TULIPANI, Medrisio (CH)

(73) Assignee: SALVATELLI srl, Montegranaro (AP) (IT)

(21) Appl. No.: 13/188,565

(22) Filed: Jul. 22, 2011

Related U.S. Application Data
(62) Division of application No. 12/336,913, filed on Dec. 17, 2008.
Application No. 12/336,913 is a continuation-in-part of application No. 11/124,026, filed on May 6, 2005, now abandoned.

(30) Foreign Application Priority Data
May 6, 2004 (IT) MC2004A000063

(51) Int. Cl. A61F 5/052

(52) U.S. Cl. 36/140

(57) ABSTRACT

The present invention is an apparatus and method of orthopedic footwear for post-surgical or diabetic patients or for sanitary use. The footwear has an ambidextrous bottom sole. In a higher part thereof, there is a groove of at least 10 mm, in which an off-loading plantar insoles or medicine or bandages can be inserted. These have been built-in the bottom sole using an ortho-dynamic concept which allows the patient a controlled deambulation. To that end, the present invention prevents sheer force on the plantar part of the bottom sole and the foot and in particular on the metatarsal and/or tarsal part of the foot.

### CROSS-REFERENCE TO RELATED APPLICATIONS

The present application is a divisional of U.S. application Ser. No. 12/336913, filed on Dec. 17, 2008, entitled “Footwear Orthosis,” presently pending. U.S. application Ser. No. 12/336913 is a continuation-in-part of U.S. application Ser. No. 11/124026, filed on 6 May 2005, entitled “Footwear Orthosis,” now abandoned.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

The present invention concerns orthopedic footwear for post-surgical or diabetic patients.

2. Description of Related Art Including Information Disclosed Under 37 CFR 1.97 and 37 CFR 1.98

In current technology, post-surgical or diabetic patients use orthopedic footwear with a modified bottom sole that makes the foot take a determined and forced inclination. Decompression is caused either only on the forefoot or only on the heel, avoiding contact of the injured part of the foot.

At this moment, post-surgical or diabetic patients use orthopedic footwear on the market which decompresses the forefoot or the heel of the patient with modifications on the front or back part of the bottom sole. A forced inclination is allowed to the foot in this way, obtaining the total absence of contact of the injured front or the back parts of the foot on the ground.

The bottom sole of this footwear is rigid and has a flat middle part, which has total contact with the ground. For three quarters thereof, the bottom sole is raised at the front or back part at an inclination of about 30 degrees.

The particular bottom sole of the above-mentioned orthopedic footwear presents the inconvenience of a difficult deambulation caused by the forced inclination of the patient's foot.

Furthermore, all of the orthopedic footwear for diabetics on the market at present, have a bottom sole with a completely flat part, which has total contact with the ground which provokes the drawback that the sheer force is concentrated in the corresponding plantar area.

A type of sole for orthopedic footwear, as described in U.S. Pat. No. 5,827,210 to Antar, is made up of a middle flat part bordered by a front part and a back part. The front part and back part are both convex towards the outer side in such a way to determine a rolling motion during the deambulation. This movement is caused by the total contact of the middle flat part of the bottom sole with the ground and by the action of the back part of the bottom sole which comes into contact with the ground by a heightened position to the ground underfoot like the front part.

The drawback of the Antar patent is that the middle flat part of the bottom sole having total contact with the surface underneath all the central plantar area is under a constant sheer force.

The aim of the present invention is to provide sanitary orthopedic footwear, for use by post-surgical or diabetic patients, produced using shock-absorbing and antibacterial material. There is a bottom sole, conceived on the ortho-dynamic concept and characterized by a ramp that allows cushioning of the shock during the landing phase of the foot, in such a way to determine a correct deambulation in the central phase of the march and a rotation in the take-offphase. This is made possible by the front and back parts of the bottom sole that are both convex to at least 20° and the part that has contact with the ground, that is only 8-12 mm long and is off-centered in correspondence to the Chopart line so as to have a constant instability of this bottom sole. This deliberate instability is necessary to avoid the concentration of the sheer force on the user's foot and in particular in the tarsal or metatarsal part.

Another aim of the present invention is to allow the use of sanitary orthopedic footwear in post-surgical or diabetic patient care, which can also be adapted to a highly-bandaged foot. The bottom sole of the present patent application, which is ambidextrous, includes two or more variations in four or more sides. The orthopedic footwear for post-surgical or diabetic patients can easily be used by patients who have different needs.

Another aim of the present invention is to provide sanitary orthopedic footwear for post-surgical or diabetic patients, characterized by a particular rigidity made possible by inserting a very rigid and crushproof insole in the inside of the bottom. This insole has a particular design and is made using antibacterial materials; it is washable and light.

Another aim of the present invention is to provide sanitary orthopedic footwear for post-surgical or diabetic patients characterized by a bottom sole having a groove in which bandages and/or medicines can be inserted. Two or more layers of particular plantar insole are characterized by modular and interchangeable elements, which can also be inserted. These allow localized sheer force reduction corresponding to diabetic foot ulcerations or plantar lesions.

These and other aims are achieved by the invention that is the subject of the present patent application relative to sanitary orthopedic footwear for post-surgical or diabetic patients in which the ambidextrous bottom sole is based on a ortho-dynamic concept, including a groove of at least lOmm in its highest structure. The off-loading plantar insole and/or medicine can be inserted in the groove. Each of the front and back parts of the bottom sole are convex with an minimum inclination of 20° with respect to the surface underneath. The only contact point with the surface underneath is off-centered on the Chopart line and is limited to 8-12 mm. For these reasons, during the deambulation the bottom sole is not, at any time, completely and continuously in contact with the surface underneath. This determines its constant inbalance, in this way avoiding sheer force on the patient's foot and in particular in the metatarsal, tarsal and central parts.

### BRIEF SUMMARY OF THE INVENTION

The present invention has an ambidextrous bottom sole. In a higher part thereof, there is a groove of at least 10 mm, in which off-loading plantar insoles or medicine or bandages can be inserted. These have been built-in using an ortho-dynamic concept which allows the patient a controlled deambulation and to that end, to avoid sheer force on the plantar part of the foot and in particular on the metatarsal and/or tarsal part.

To avoid the above-mentioned drawbacks of the prior art, the bottom sole of the orthopedic footwear, which is the subject of the present patent, has only a single point of contact, being only a few millimeters long (8-12 mm) with the surface underneath. It has a highly defined convexity at the front and the back (a minimum of 20° inclination in respect to the surface underneath). Furthermore the point of contact with the ground in the static phase has been deliberately placed off-centered in correspondence to the Chopart line of the foot.

The Chopart line is the defined neutral line that separates the anklebone scaphoid and the heel cuboid joints, statistical studies have shown the lowest possibility of diabetic plantar lesions on this line.

### BRIEF DESCRIPTION OF THE SEVERAL VIEWS OF THE DRAWINGS

Other characteristics and advantages of the invention will result from a form of implementation of the present invention, preferred but not exclusively sanitary orthopedic footwear for post-surgical or diabetic patients in medical use. The present patent application is illustrated approximately in the drawings.

FIGS. 1, 1/a, and 1/b are side elevation views of the bottom sole in the three positions from the deambulation phase of the sanitary orthopedic footwear for post-surgical or diabetic patients.

FIG. 2 is an exploded elevation view of the bottom sole of the footwear of the present patent application, showing the rigid insert insole and the three-plantar insole system that are removed from the bottom sole.

FIG. 3 is a top plan view of one of the three modular and interchangeable plantar insoles that make up the plantar insole system.

FIG. 4 is another side elevation view of the orthopedic footwear of the present invention.

FIGS. 5A and 5B are also side elevation views of the orthopedic footwear of the present invention.

### DETAILED DESCRIPTION OF THE INVENTION

The rigidity of the bottom sole is obtained by an element called a “rigid insole”, which is inserted into the bottom sole in an opposite groove, having a function of avoiding flexing and torsion in the entire plantar area of the foot, both in a static or dynamic phase. This rigid insole is extremely resistant to repeated flexes, moulded with a very rigid, light, uncrushable, antibacterial and washable material. The molds are the exclusive property of the company that is presenting this patent application. This rigid insole is characterized by a particular rigidity, lightness and uncrushability, and it has a completely flat structure on the upper part which is in contact with the foot and lower structure that follows the lines of the bottom sole to which it is attached. This is to ensure that the foot rests on a completely flat surface without modifying the deambulation created by the ramp of the bottom of the present patent application.

Orthopedic footwear for diabetic patients must allow the total decompression ofthe patient's foot where the diabetic plantar ulceration is present. To achieve this result, the diabetic footwear that has a bottom sole that has the shape and characteristics as described above, offering the possibility to insert a series of three multi-level, washable off-loading plantar insole with different mechanical and shock-absorbent characteristics for each layer in the groove of the sole bottom. Each of these plantar insoles is characterized by three modular and exchangeable systems, being used to change hardness and softness in such a way to guarantee the total off-load of the injured part of the foot according to the patient's needs.

All of the orthopedic footwear has uses for either diabetic patients or sanitary use, being available on the market at present and being removable by the patients themselves.

As many scientific studies have demonstrated that irremovable off-loading devices for diabetic foot lesions shorten considerably the healing time of lesions, by reducing patient's non-compliance during the traitment, the present invention includes a non-removable system which consists of the application of inserts for the passage of disposable self-blocking strips that, once sealed, do not allow patient to reopen the footwear and remove it.

Non-removable system consists of two inserts of flexible semi-rigid material with a eyelet in the middle, anchored at the external upper sides of the upper. Theese inserts allow the insertion of a plastic strip with disposable pressure fastening system (of the kind commonly available on market) which is provided with the device.

This kind of strip in soft and flexible material passes through the holes of the two lateral inserts and, once closed on its opposite extremity, the device will be blocked at the minimum height of the ankle or at the maximum height of the shin-calf and it cannot longer be reopened or removed if not by cutting it by means of proper cutting tool.

Non-removable systems allow a physician to fix the sanitary orthopedic footwear for diabetic patients or sanitary use to the patient's foot. The non-compliant patient could remove the orthopedic footwear from his/her foot independently.

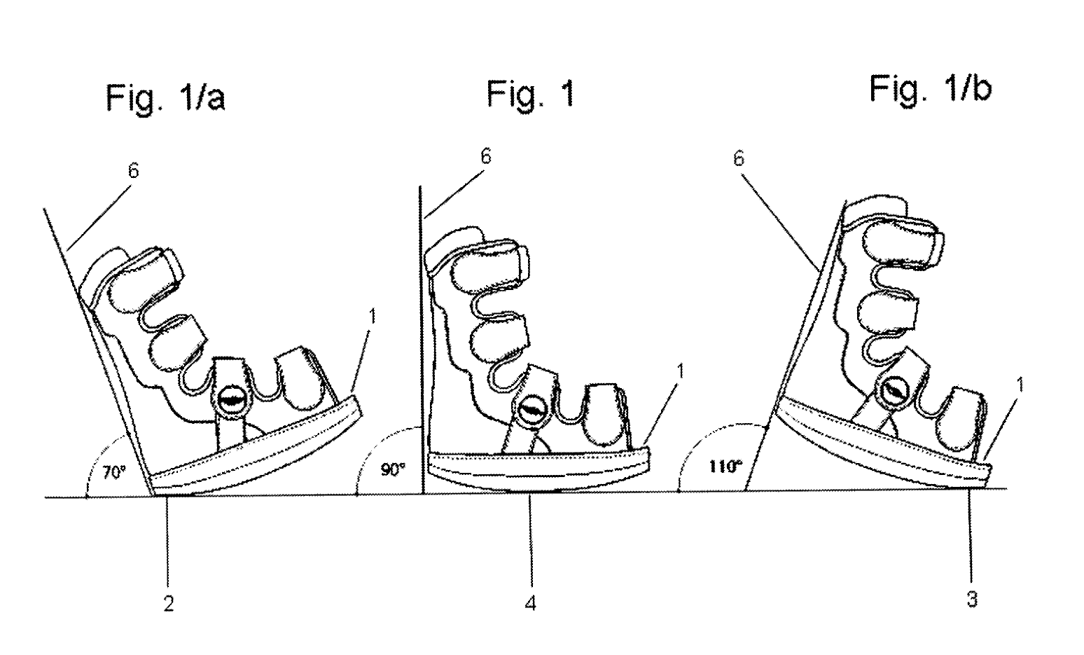

In FIGS. 1, 1/a and 1/b, the bottom sole 1 is shown with a deambulation ramp with an inclination of 70° when the foot lands on the back part (2) (FIG. 1/a) and 110° in the taking off phase on the front part (3) (FIG. 1/b). In the static phase, the point of contact with ground (4) of the bottom sole (1) has an inclination of 90° off-centered in the Chopart line (FIG. 1). The bottom sole (1) is characterized by the fact in the deambulation phase its inclination goes from 70° of the back part (2) to an inclination of 90° in the central part (4) which increases up to 110° at the front part (3).

Furthermore, the front (3) and back (2) parts are both convex with the same inclination of 20° with respect to the surface underneath and the contact point (4), that is off-centered from the Chopart line (6), is limited to 8-12 mm. These characteristics ensure the bottom sole is never completely and continuously in contact with the surface underneath, causing a constant inbalance of the contact point. The bottom sole ensures that the concentration of sheer force is not present on the patient's foot especially on the tarsal, metatarsal and central parts.

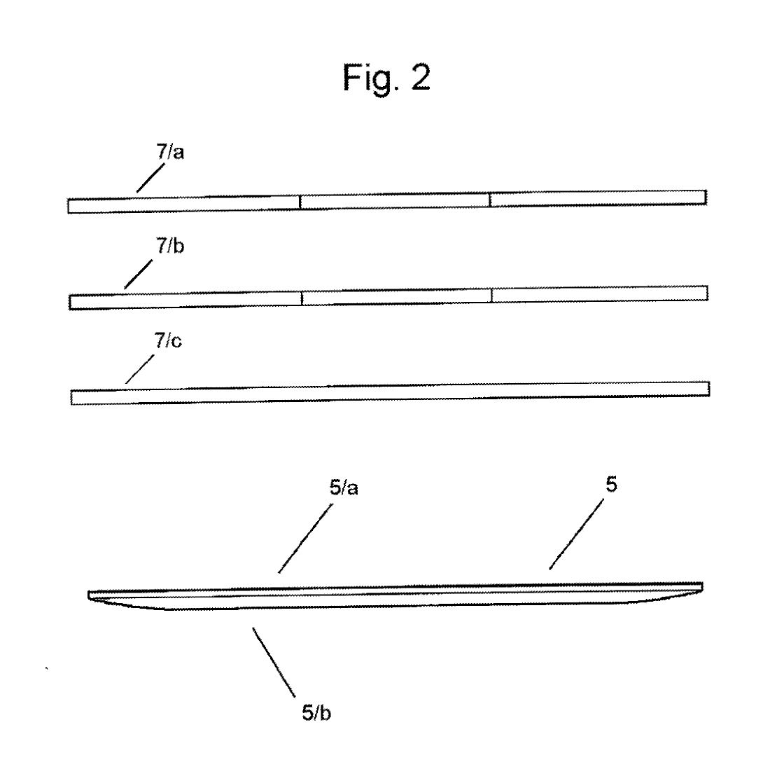

To achieve this result, it is also necessary that the bottom sole (1) is rigid to avoid its flexions and torsions. A rigid insole (5) is inserted into the bottom sole groove. The rigid insole (5) also shown in FIG. 2, is made with antibacterial, washable material and is characterized by a high resistance to repeated flexions, lightness and its uncrushability. This rigid insole (5) is characterized by a structure that permits a flat higher surface 5/a that has contact with the foot and one lower surface 5/b that follows the shape of the bottom sole to which it is attached. In such a way, it does not alter the deambulation created by the ramp of the self-same bottom sole.

Another component of the bottom sole of the above mentioned type of orthopedic footwear is the series of two or more multilayer plantar insoles (7/a), (7/b), (7/c) in FIG. 2, with mechanical and shock-absorbent characteristics with different characteristics and that are produced using washable and disinfectable materials.

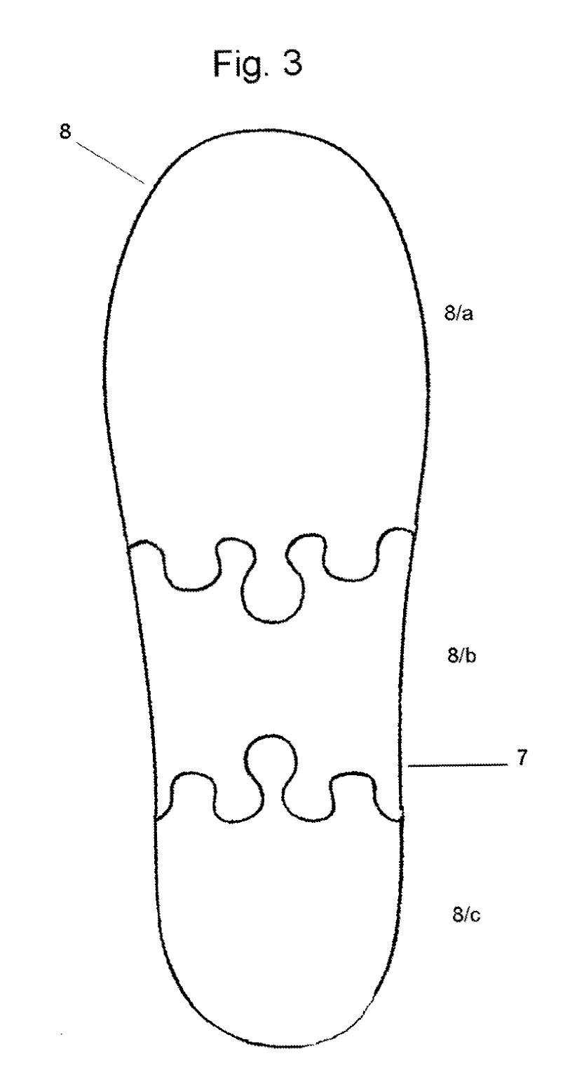

FIG. 3 is a view one of the two or more plantar insoles described in the previous paragraph seen from above, each of which is made up of two or three modular elements and which are interchangeable between the three pieces. The forefoot (8/a) is represented as a part thereof. The central plantar insole (8/b) is another part, and back part (8/c) is represented by the back part of the plantar insole.

This series of two or more modular plantar insoles (7/a), (7/b) and (7/c), each of which is made up of one element with three modular and interchangeable elements (8/a), (8/b) and (8/c) makes it possible to alter the hardness and elasticity according the patients' needs and to guarantee the total off-load of the diabetic foot ulceration or plantar lesions.

The bottom sole that is the subject of this patent application is furthermore characterized by a ambidextrous shape that allows us to create post-surgical footwear that can be used either by diabetic patients or for sanitary use in two or more variations and in four or more lengths and so it can be easily used according to the various needs of the patients

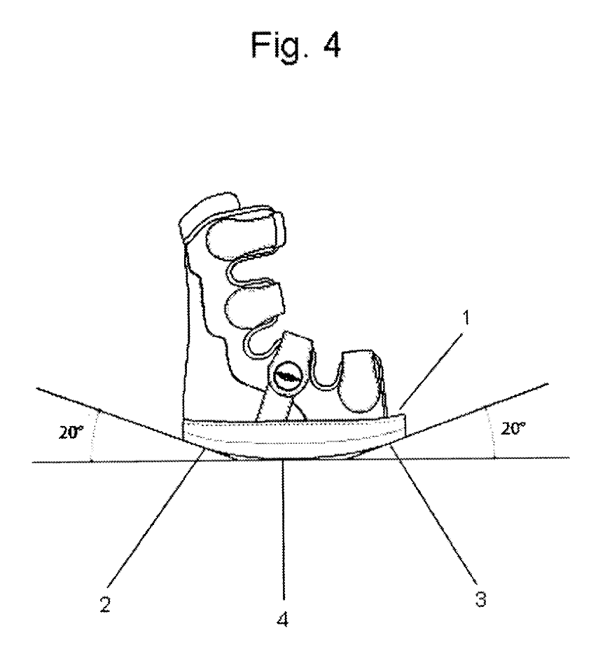

FIG. 4 shows the elevation view of the footwear again, at the front and back parts of the bottom sole, being convex each with an inclination of 20° and with the middle contact point limited to 8-12 mm.

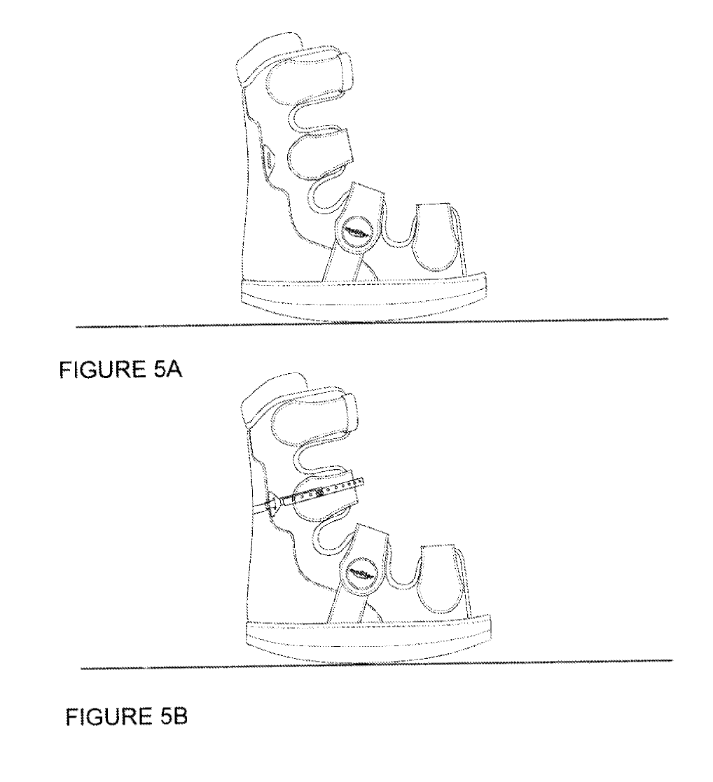

FIG. 5a shows a view of one of the two external lateral parts of the upper of the orthopedic footwear for either diabetics or sanitary use. The insert in a semi-rigid material with a eyelet in the middle, anchored at the external upper sides thereof, necessary for the passage of disposable self-blocking strips.

FIG. 5b represents an external lateral view of the upper sides, characterized by the non-removable system equipped with disposable self-blocking strips, that pass through the flexible and semi-rigid insert eyelets anchored at the external upper parts of the upper sides. After having been completely sealed, the orthopedic footwear for the patient's foot will be cut by means of a proper cutting tool.

The invention thus conceived can be subjected to many modifications and variants, all of these enter the sphere of the invented concept, furthermore the materials and sizes of the above mentioned invention, illustrated in the accompanying designs and later laid claim to, can be made according to the needs.

We claim:

1. A method of using orthopedic footwear for post-surgical or diabetic patients or for sanitary use, the method comprising the steps of:
mounting an ambidextrous bottom sole with a groove, said groove being positioned in an upper part of the bottom sole and being at least 10 mm in length;
housing an off-load plantar insole or medicine in said groove; and
inclining the bottom sole from a front part to a back part back and forth, the front and back parts being convex with a minimum inclination, in relation to the ground underneath, the parts being convex at an inclination of 20° and having a point of contact, off-centered on the Chopart line limited to 8-12 mm, the bottom sole avoiding complete and continuous contact with the surface underneath so as to prevent a sheer force in a deambulation phase and in particular on portions of the bottom sole corresponding to the metatarsal, tarsal and central areas.

2. The method of using orthopedic footwear according to claim 1, said step of inclining the bottom sole comprises inclining a deambulation ramp at 70° in a landing phase of the back part, inclining the deambulation ramp at 90° when off-centered on the Chopart line, and inclining the deambulation ramp at 110° in a take-off phase.

3. The method of using orthopedic footwear according to claim 2, wherein said deambulation ramp inclines from 70° on the back part, to 90° on a central part and then increasing to 110° on the front part.

4. The method of using orthopedic footwear according to claim 1, said off-load plantar insole is rigid, molded, light and deformable, avoiding flexing and torsion and consequently any unwanted weight on a plantar part of the bottom sole.

5. The method of using orthopedic footwear according to claim 4, wherein the rigid plantar insole is characterized by a particular shape or nature, providing a flat surface in contact with said bottom sole and a surface, following a shape of the bottom sole without affecting the step of inclining back and forth.

6. The method of using orthopedic footwear according to claim 2, wherein said off-load plantar insole is comprised of a modular series of two or more multi-layer plantar insoles with mechanical and shock-absorbent characteristics, being comprised of washable and disinfectable materials.

7. The method of using orthopedic footwear according to claim 1, wherein said off-load plantar insole is comprised of at least two multi-layer plantar insoles, each multilayer plantar insole being comprised of three modular and interchangeable elements with variable hardness and elasticity.

8. The method of using orthopedic footwear according to claim 1, wherein said bottom sole has two or more variants or four or more lengths.

9. The method of using orthopedic footwear according to claim 1, wherein said upper part of the bottom sole comprises two external upper side parts, two inserts in semi-rigid material with a eyelet, and a strip, said eyelet placed in a middle of each insert, said strip being inserted into said eyelet, said upper part further comprising a disposable sealing means, locking said strip.
